(19) United States
(12) Patent Application Publication
(10) Pub. No.: US 2015/0018704 A1
(43) Pub. Date: Jan. 15, 2015

(54) REAL-TIME MULTI-CHANNEL AUTOMATIC EYE BLINK ARTIFACT ELIMINATOR

(71) Applicant: National Chiao Tung University, Hsinchu (TW)

(72) Inventors: Wai-Chi Fang, Hsinchu (TW); Jui-Chieh Liao, Hsinchu (TW); Wei-Yeh Shih, Hsinchu (TW); Kuan-Ju Huang, Hsinchu (TW); Chiu-Kuo Chen, Hsinchu (TW)

(73) Assignee: National Chiao Tung University, Hsinchu (TW)

(21) Appl. No.: 14/099,523

(22) Filed: Dec. 6, 2013

(30) Foreign Application Priority Data
Jul. 10, 2013 (TW) 102124669

(51) Int. Cl. A61B 5/00; A61B 5/0476; A61B 5/11; A61B 5/04

(52) U.S. Cl. CPC A61B 5/7214; A61B 5/04012; A61B 5/0476; A61B 5/1103
USPC 600/544

(57) ABSTRACT

A real-time multi-channel automatic eye blink artifact eliminator includes a receiving unit for receiving independent component data; a temporary storage unit for saving pieces of the independent component data to form a data segment; a detection unit for detecting an eye blink artifact in the data segment through a sample entropy algorithm to generate a sample entropy value corresponding to the data segment; and a processing unit for determining whether the data segment contains the eye blink artifact according to the sample entropy value to generate an output result, eliminating the eye blink artifact according to the output result and outputting processed independent component data. The receiving unit continuously receives a next piece of the independent component data and the temporary storage unit discards the oldest one and adds a new one to form a new data segment, thereby continuously performing the eye blink artifact elimination to each data segment.

### BACKGROUND OF THE INVENTION

1. Field of the Invention

The present invention relates to elimination of eye blink artifacts from EEG (electroencephalogram) signals, and more particularly, to a real-time multi-channel automatic eye blink artifact eliminator based on multi-channel EEG processing.

2. Description of Related Art

The human brain emits electromagnetic waves (i.e., brain waves) and the brain waves can be recorded by an EEG. By measuring potential changes of nerve cells in the brain, abnormal electrical discharges or potentials in the brain can be determined. Further, medical diagnosis such as diagnoses of epilepsy, central nervous system abnormalities, dementia and so on can be performed based on measurement of brain waves.

During measurement of brain waves, multiple channels are generally used to improve spatial resolution. Further, independent component analysis effectively separates independent component signals and noises. For example, an eye blink artifact is a kind of noise that is induced by movement of eyes and needs to be eliminated during the independent component analysis. In a conventional automatic eye blink detection method, a large amount of data need to be collected before operation and accordingly a large amount of waiting time is consumed before data processing and output. Consequently, EEG signals without eye blink artifacts cannot be timely provided to an application terminal. To overcome the drawback, people reduce the amount of data to be collected as well as the amount of waiting time, which however easily leads to an insufficient amount of data for operation. Further, if an eye blink artifact is located at a boundary between data, an error may occur in the elimination of the eye blink artifact. A sliding window method may be used to overcome the boundary drawback, which however cannot achieve a fast operation.

The above-described methods need long waiting time or easily result in a determination error. Specifically, it is difficult to eliminate eye blink artifacts in the independent component analysis of a portable medical instrument since a large amount of operations and high hardware complexity are required in the elimination.

Therefore, there is a need to provide a real-time multi-channel automatic eye blink artifact eliminator implemented with hardware so as to provide real-time and correct EEG signals.

### SUMMARY OF THE INVENTION

In view of the above-described drawbacks, the present invention provides a real-time multi-channel automatic eye blink artifact eliminator that is implemented with a VLSI hardware.

The real-time multi-channel automatic eye blink artifact eliminator comprises: a receiving unit for receiving independent component data in a channel, wherein the independent component data represent EEG signals in a time segment; a temporary storage unit for temporarily storing multiple pieces of the independent component data from the receiving unit, wherein the multiple pieces of the independent component data form a data segment according to the time segment; a detection unit for detecting an eye blink artifact in the data segment through a sample entropy algorithm so as to generate a sample entropy value corresponding to the data segment in the time segment; and a processing unit for determining whether the data segment contains the eye blink artifact according to the sample entropy value so as to generate an output result, eliminating the eye blink artifact according to the output result and outputting processed independent component data representing the data segment, wherein the receiving unit continuously receives a next piece of the independent component data such that the temporary storage unit discards the oldest piece of the independent component data in the data segment and adds a new piece of the independent component data so as to form a new data segment, and the new data segment is further processed by the detection unit and the processing unit, thereby continuously performing the eye blink artifact elimination to each data segment.

In an embodiment, the temporary storage unit stores the multiple pieces of the independent component data in a FIFO (first in first out) manner so as to form the data segment, and the detection unit starts to detect the eye blink artifact when the number of the pieces of the independent component data in the data segment reaches 128.

In an embodiment, if the data segment contains more than a half of the waveforms of the eye blink artifact, the processing unit determines that the data segment contains the eye blink artifact.

In an embodiment, if the sample entropy value is less than a predetermined threshold value, the processing unit determines that the data segment contains the eye blink artifact.

The processing unit outputs a value of zero as the output result for the data segment that contains the eye blink artifact, and the processing unit outputs the 96^{th} piece of the independent component data as the output result for the data segment that does not contain the eye blink artifact.

In an embodiment, the processing unit sequentially performs the eye blink artifact elimination to data segments of multiple channels in the same time segment. By performing an inverse independent component analysis, the processed independent component data outputted by the processing unit can be converted into EEG signal data without the eye blink artifact for a subsequent EEG signal reconstruction.

Therefore, the real-time multi-channel automatic eye blink artifact eliminator of the present invention is used in combination with an independent component analysis processor so as to timely and automatically remove eye blink artifacts from independent component data of EEG signals. As such, processed EEG signals without the eye blink artifacts can be timely provided to back-end applications. Also, the present invention implements an effective and real-time operation under multi-channel and real-time specifications and overcomes the conventional determination error occurring when an eye blink artifact is present at a boundary. Therefore, the real-time multi-channel automatic eye blink artifact eliminator of the present invention can be implemented with a VLSI hardware and used to eliminate eye blink artifacts from EEG signals so as for the EEG signal reconstruction. The present invention can be applied to portable medical instruments so as to provide real-time and correct EEG signals, which are helpful to family care and medical treatment.

### BRIEF DESCRIPTION OF DRAWINGS

FIG. 1 is a schematic diagram showing processing and reconstruction of EEG signals;

FIG. 2 is a schematic block diagram of a real-time multi-channel automatic eye blink artifact eliminator of the present invention;

FIG. 3 is a schematic diagram showing data processing through a cache memory of the real-time multi-channel automatic eye blink artifact eliminator of the present invention;

FIG. 4 is a schematic flow diagram showing processing of eye blink artifacts through the real-time multi-channel automatic eye blink artifact eliminator of the present invention;

FIG. 5 is a schematic diagram showing the hardware structure of the real-time multi-channel automatic eye blink artifact eliminator of the present invention;

FIGS. 6A and 6B are schematic diagrams showing determination of boundary artifacts by the real-time multi-channel automatic eye blink artifact eliminator of the present invention; and

FIGS. 7A and 7B are schematic diagrams showing independent component data of EEG signals before and after elimination of eye blink artifacts by the real-time multi-channel automatic eye blink artifact eliminator of the present invention.

### DETAILED DESCRIPTION OF PREFERRED EMBODIMENTS

The following illustrative embodiments are provided to illustrate the disclosure of the present invention, these and other advantages and effects can be apparent to those in the art after reading this specification.

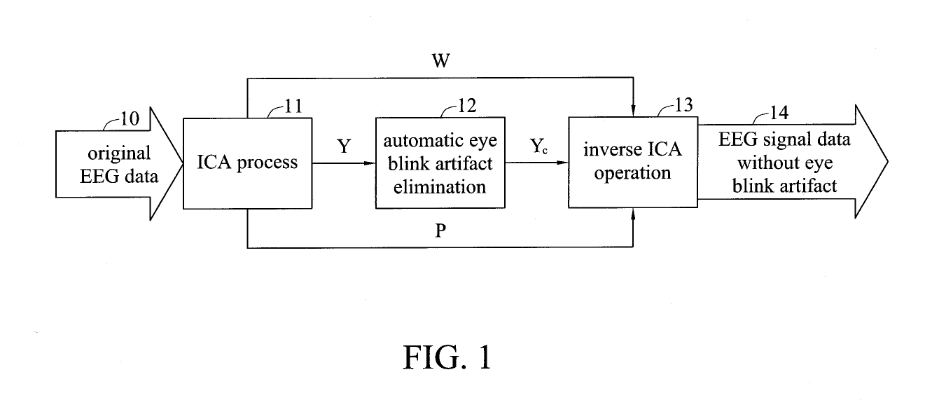

FIG. 1 is a schematic diagram showing processing and reconstruction of EEG signals. Referring to FIG. 1, the present invention performs elimination of eye blink artifacts between ICA (independent component analysis) operation and reconstruction of EEG signals through a real-time multi-channel automatic eye blink artifact eliminator so as to eliminate ICA processing results with eye blink artifacts. Then, an inverse ICA operation is performed to reconstruct EGG signals without eye blink artifacts for back-end applications.

In particular, original EEG data 10 are obtained through detection and an ICA process 11 is performed to the original EEG data 10. That is, through an online recursive independent component analysis of a real-time multi-channel EEG signal processor, sampling signals are whitening processed by a whitening matrix P and independent component analyzed by a demixing matrix W so as to generate independent component data Y. Then, a process of automatic eye blink artifact elimination 12 is performed to the independent component data Y to generate data Yc. That is, the process determines whether the independent component data Y contain an eye blink artifact, and if necessary, the process eliminates the eye blink artifact to generate the processed independent component data Yc. Thereafter, an inverse ICA operation 13 is performed to the independent component data Yc to generate EEG signal data 14 without the eye blink artifact for reconstruction of EEG signals. Therefore, the eye blink artifact elimination is performed by the real-time multi-channel automatic eye blink artifact eliminator of the present invention between an ICA operation and reconstruction of EEG signals.

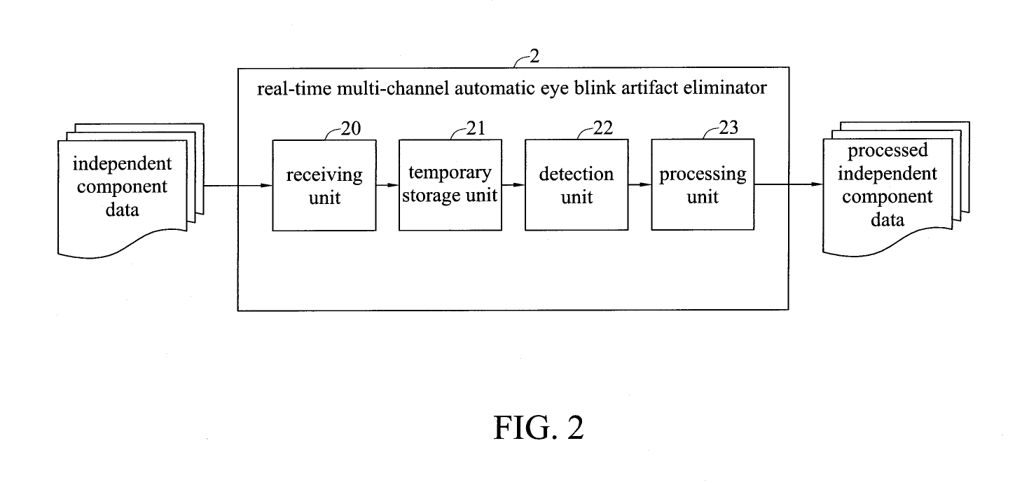

FIG. 2 is a block diagram of the real-time multi-channel automatic eye blink artifact eliminator of the present invention. Referring to FIG. 2, the real-time multi-channel automatic eye blink artifact eliminator 2 includes a receiving unit 20, a temporary storage unit 21, a detection unit 22 and a processing unit 23.

The receiving unit 20 is used to receive independent component data in a channel. The independent component data represent EEG signals in a time segment. Since multiple channels can be used to achieve EEG signals with high spatial resolution, each channel generates independent component data. The real-time multi-channel automatic eye blink artifact eliminator 2 collects the independent component data of each channel for processing. In the present embodiment, one channel is exemplified for description. Further, the independent component data received by the receiving unit 20 is generated by an independent component analysis processor (i.e., the above-described real-time multi-channel EEG signal processor) after performing an independent component analysis.

The temporary storage unit 21 is used to temporarily store multiple pieces of the independent component data from the receiving unit 20. The multiple pieces of the independent component data sequentially form a data segment according to the time segment. The multiple pieces of the independent component data are obtained by continuously capturing and processing EEG signals with the time segment. The independent component data in the channel sequentially form a data segment with time. That is, the data segment represents the continuous EEG signals obtained by the channel.

The detection unit 22 is used to detect an eye blink artifact in the data segment through a sample entropy algorithm, thereby generating a sample entropy value corresponding to the data segment in the time segment. In particular, by sampling EEG signals in a fixed time segment, the detection unit detects an eye blink artifact in the EEG signals through a sample entropy algorithm so as to generate a sample entropy value that serves as a base for determining whether the data segment contains the eye blink artifact.

The processing unit 23 is used to determine whether the data segment contains the eye blink artifact according to the sample entropy value so as to generate an output result and eliminate the eye blink artifact according to the output result and output processed independent component data representing the data segment. In particular, the processing unit 23 determines whether the EEG signals contain the eye blink artifact according to the sample entropy value. If it is determined that the data segment contains the eye blink artifact, the data segment is eliminated and a value of zero is outputted as the output result. Otherwise, if it is determined that the data segment does not contain the eye blink artifact, the 96^{th} piece of the independent component data in the data segment is outputted as the output result. In other words, the output result represents whether it is performed to eliminate the eye blink artifact.

In an embodiment, the detection unit 23 starts to detect the eye blink artifact when the number of the pieces of the independent component data in the data segment reaches 128, which is associated with the sampling rate and number. Conventionally, 128 pieces of the independent component data must be collected before making a determination, which results in a waiting process. Compared with the prior art, the real-time multi-channel automatic eye blink artifact eliminator 2 of the present embodiment makes a determination once the number of the pieces of the independent component data reaches 128. Thereafter, each time a new piece of the independent component data is received, a new data segment is generated through data replacement and it is determined immediately whether the new data segment contains the eye blink artifact.

Therefore, the receiving unit 20 continuously receives a next piece of the independent component data and the temporary storage unit 21 discards the oldest piece of the independent component data in the data segment and adds the new piece of the independent component data in a FIFO (first in first out) manner, thus forming a new data segment. The new data segment is further processed by the detection unit 22 and the processing unit 23, thereby continuously performing the eye blink artifact elimination to each data segment and generating processed independent component data representing the data segment. That is, since the next piece of the independent component data is continuously obtained, the present embodiment can continuously form a new data segment and immediately determine whether the data segment contains the eye blink artifact, thus dispensing with the need to wait for collection of 128 pieces of data as in the prior art.

Further, how to determine whether a data segment contains the eye blink artifact is described as follows. Generally, if a data segment contains more than a half of the waveforms of the eye blink artifact, it can be determined that the data segment contains the eye blink artifact. In other words, the processing unit 23 determines that the data segment contains the eye blink artifact when the data segment contains more than a half of the waveforms of the eye blink artifact. To facilitate determination in hardware implementation, all detection and determination are numerically represented. The detection unit 22 generates a sample entropy value corresponding to the data segment in a certain time segment and the processing unit 23 compares the sample entropy value with a predetermined threshold value. If the sample entropy value is less than the predetermined threshold value, the processing unit 23 can determine that the data segment contains the eye blink artifact.

The above-described data collection and eye blink artifact detection and elimination are associated with the independent component data of a single channel. The processing unit 23 needs to perform the eye blink artifact elimination to data segments of the multiple channels so as to reconstruct EEG signals in multiple channels.

Finally, an inverse ICA operation is performed to the processed independent component data so as to form EEG signal data without the eye blink artifact for subsequent EEG signal reconstruction.

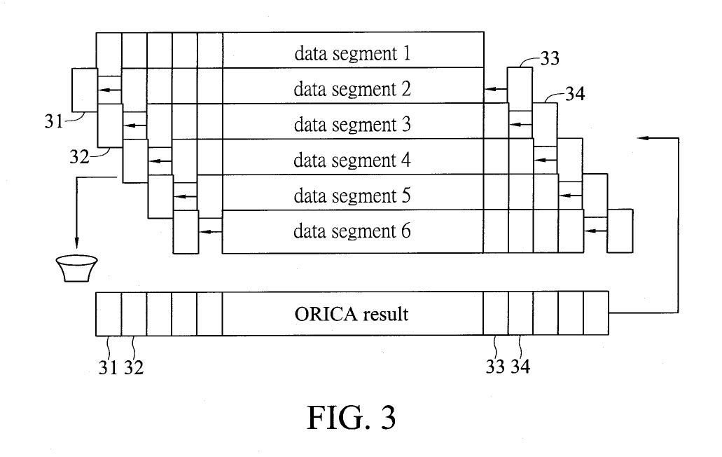

FIG. 3 is a schematic diagram showing data processing through a cache memory of the real-time multi-channel automatic eye blink artifact eliminator of the present invention. As described above, when a next piece of the independent component data is received, the oldest piece of the independent component data in the data segment is discarded and the new piece of the independent component data is added in a FIFO manner.

Referring to FIG. 3, the bottom data set is the result from an online recursive independent component analysis (ORICA). Therein, the independent component data becomes newer towards the right side and older towards the left side. Data segment 1 contains the oldest piece of the independent component data 31, the second oldest piece of the independent component data 32 and so on. When a new piece of the independent component data 33 is received, the oldest piece of the independent component data 31 is discarded and the new piece of the independent component data 33 is added to form data segment 2. In the same manner, when a next new piece of the independent component data 34 is received, the present oldest piece of the independent component data 32 is discarded and the new piece of the independent component data segment 34 is added to form the data segment 3. As such, a new data segment is formed based on an FIFO manner and an eye blink artifact determination can be immediately made to the new data segment.

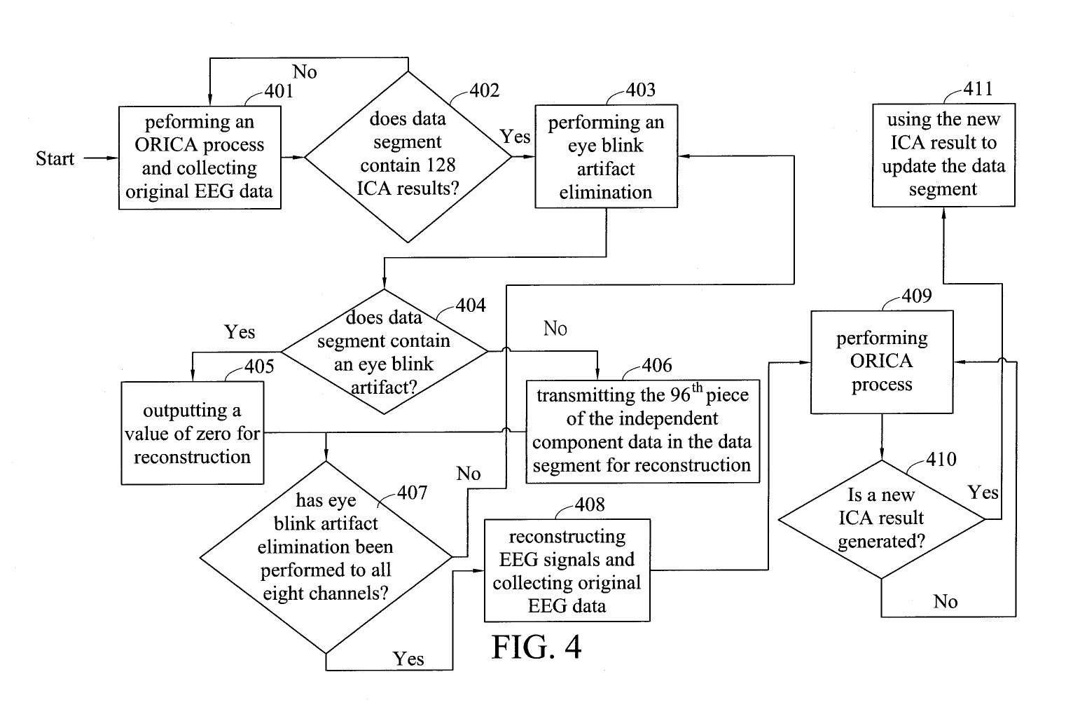

FIG. 4 is a flow diagram showing processing of eye blink artifacts through the real-time multi-channel automatic eye blink artifact eliminator of the present invention. Referring to FIG. 4, at first, EEG signals are received. Then, eye blink artifacts are eliminated for EEG signal reconstruction. During the process, new EEG signals are continuously received.

At step 401, original EEG data are collected, and an ORICA process such as the above-described whitening process is performed to the original EEG data so as to generate independent component data. Then, the process goes to step 402.

At step 402, it is determined whether there are 128 pieces of the independent component data in a data segment. As described above, the first determination is performed after 128 pieces of the independent component data are collected. If there are not 128 pieces of the independent component data, the process goes to step 401. Otherwise, the process goes to step 403.

At step 403, an eye blink artifact elimination is performed. Then, at step 404, it is determined whether the data segment contains an eye blink artifact. If yes, the process goes to step 405 to generate a value of zero for reconstruction. The value of zero represents the data segment contains the eye blink artifact and cannot be used. Otherwise, the process goes to step 406 to transmit the 96^{th} piece of the independent component data of the data segment for reconstruction.

The reason that the 96^{th} piece of the independent component data of the data segment is used to represent the data segment is described as follows. If a half of waveforms of the eye blink artifact are present in the data segment, it is determined that the data segment contains the eye blink artifact. Therefore, it is already determined whether the data segment contains the eye blink artifact at the 96^{th} piece of the independent component data. Therefore, the eye blink artifact elimination of the present invention uses the 96^{th} piece of the independent component data to represent the data segment.

Thereafter, at step 407 that follows step 405 or 406, it is determined whether the eye blink artifact elimination has been performed on all eight channels. As described above, the present invention relates to multi-channel EEG processing. EEG signals in each channel in the same time segment need to be processed before processing EEG signals in the next time segment. Therefore, if the eye blink artifact elimination has not been performed on all channels, the process goes back to step 403 for performing the eye blink artifact elimination to the channels. Otherwise, the process goes to step 408.

At step 408, EEG signals are reconstructed and original EEG data are collected. That is, when EEG signals are reconstructed, new original EEG data are continuously collected. Then, the process goes to step 409.

At step 409, an ORICA process is performed to the new original EEG data. Then, at step 410, it is determined whether a new ICA result, i.e, a new piece of the independent component data, is generated. If not, the process goes back to step 409. Otherwise, the process goes to step 411.

At step 411, the new ICA result is used to update the data segment. That is, the new piece of the independent component data is added to the data segment and the oldest piece of the independent component data in the data segment is eliminated so as to form a new data segment. Then, the process goes back to step 403 for performing the eye blink artifact elimination to the new data segment. Therefore, when new piece of the independent component data is generated, the eye blink artifact determination and elimination are performed.

Therefore, when a new piece of the independent component data is inputted, the eye blink artifact elimination is immediately performed. The new piece of the independent component data is added to the data segment in FIFO and the oldest piece of the independent component data is discarded. As such, the 96^{th} piece of the independent component data is inputted if it is determined that the data segment does not contain the eye blink artifact. Therefore, the data segment only results in a 0.25s delay for completing the eye blink artifact detection. Compared with the prior art that requires collection of 128 pieces of data each time before performing an eye blink artifact elimination, the present invention is real-time, effective and applicable in subsequent back-end applications.

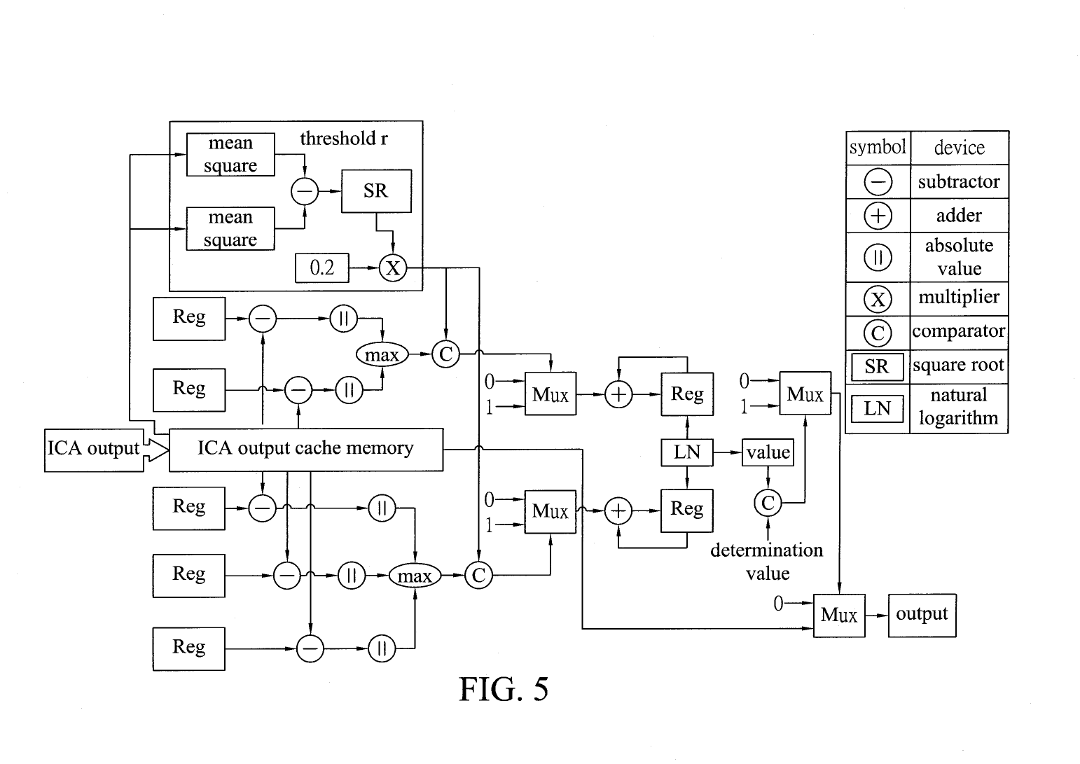

FIG. 5 shows the hardware structure of the real-time multi-channel automatic eye blink artifact eliminator of the present invention. Referring to FIG. 5, independent component data outputted from an independent component analysis (ICA) are stored in an ICA output cache memory and form a data segment. The block indicating the threshold r is used to detect an eye blink artifact on a data segment of the ICA output cache memory through a sample entropy algorithm so as to generate a sample entropy value corresponding to the data segment. Then, the sample entropy value is transmitted to a comparator and compared with a predetermined threshold so as to determine whether the data segment contains the eye blink artifact. Since such a hardware structure is well known in the art, detailed description thereof is omitted herein.

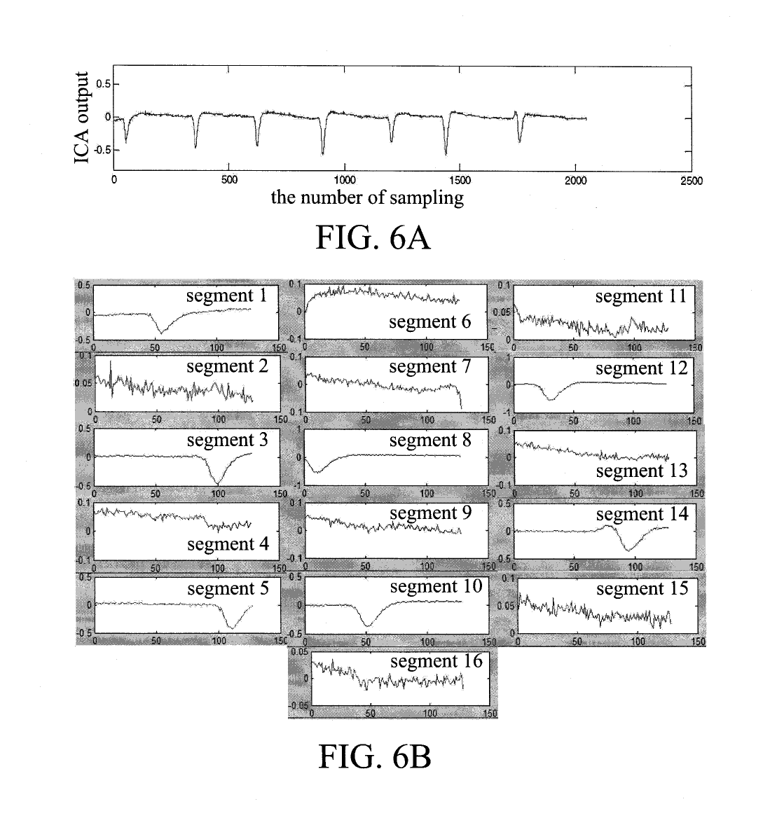

FIGS. 6A and 6B show determination of boundary artifacts by the real-time multi-channel automatic eye blink artifact eliminator of the present invention. As described above, in the prior art, it is difficult to determine an eye blink artifact that is present at a boundary. The present invention determines the data segment contains an eye blink artifact if the data segment contains more than a half of waveforms of the eye blink artifact, which is detailed as follows.

FIG. 6A shows independent component data of EEG signals in a single channel. Referring to FIG. 6A, the independent component data include a plurality of portions that protrude downward, which refer to eye blink artifacts. The EEG signals are divided into 16 segments, as shown in FIG. 6B. Each of the segments may or may not contain an eye blink artifact. Referring to FIG. 6B, it is evident that segments 1, 3, 5, 8, 10, 12, 14 contain eye blink artifacts. However, the right side of segment 7 and the left side of segment 6 only contain a portion of eye blink artifacts. As such, it is difficult to determine whether the segments contain eye blink artifacts. Therefore, by determining whether the sample entropy value is less than a predetermined threshold value, the invention can determine whether the EEG signals contains eye blink artifacts.

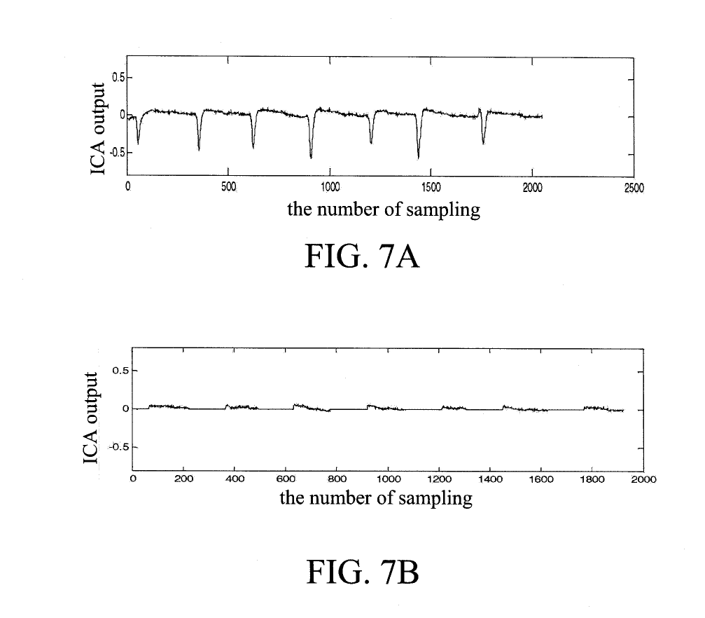

FIGS. 7A and 7B show independent component data of EEG signals before and after elimination of eye blink artifacts by the real-time multi-channel automatic eye blink artifact eliminator of the present invention. Referring to FIG. 7A, the independent component data outputted from an independent component analysis contain eye blink artifacts, i.e., the portions protruding downwards. If it is determined that a data segment contains an eye blink artifact, a value of zero is used to represent EEG signals. As such, the protruding portions of FIG. 7A are replaced by a value of zero. FIG. 7B shows the result after the eye blink artifacts are eliminated.

In an embodiment, according to the size, cost, effect etc., the real-time multi-channel automatic eye blink artifact eliminator can be fabricated in a chip based on, for example, TSMC 90 nm CMOS technology. The chip has an area of 320×320 um² and an operating frequency of 10 MHz. A Nanosim simulation shows that the chip has a power consumption of 0.138 mW at a supply voltage of 1.0V and an operating frequency of 10 MHz. Such chip can be applied in a portable EEG measurement apparatus.

Therefore, the real-time multi-channel automatic eye blink artifact eliminator of the present invention can be used in an independent component analysis so as to remove eye blink artifacts from EEG signals. According to the present invention, after an eye blink artifact determination is started, new EEG signals are continuously received. As such, when a new piece of the independent component data is added to a data segment, the oldest piece of the independent component data in the data segment is discarded, thus implementing real-time and automatic detection and elimination of eye blink artifacts. Further, the present invention overcomes the conventional determination error occurring when an eye blink artifact is present at a boundary, and facilitates an independent component analysis of EEG signals in multiple channels. The present invention implements the real-time multi-channel automatic eye blink artifact eliminator with VLSI hardware to eliminate eye blink artifacts from EEG signals so as for EEG signal reconstruction. The present invention can be applied in portable medical instruments to facilitate family care and medical treatment.

The above-described descriptions of the detailed embodiments are only to illustrate the preferred implementation according to the present invention, and it is not to limit the scope of the present invention. Accordingly, all modifications and variations completed by those with ordinary skill in the art should fall within the scope of present invention defined by the appended claims.

What is claimed is:

1. A real-time multi-channel automatic eye blink artifact eliminator, comprising:
a receiving unit for receiving independent component data in a channel, wherein the independent component data represent EEG signals in a time segment;
a temporary storage unit for temporarily storing multiple pieces of the independent component data from the receiving unit, wherein the multiple pieces of the independent component data form a data segment according to the time segment;
a detection unit for detecting an eye blink artifact in the data segment through a sample entropy algorithm so as to generate a sample entropy value corresponding to the data segment in the time segment; and
a processing unit for determining whether the data segment contains the eye blink artifact according to the sample entropy value so as to generate an output result, eliminating the eye blink artifact according to the output result and outputting processed independent component data representing the data segment,
wherein the receiving unit continuously receives a next piece of the independent component data such that the temporary storage unit discards the oldest piece of the independent component data in the data segment and adds a new piece of the independent component data so as to form a new data segment, and the new data segment is further processed by the detection unit and the processing unit, thereby continuously performing the eye blink artifact elimination to each data segment.

2. The eliminator of claim 1, wherein the temporary storage unit stores the multiple pieces of the independent component data in a FIFO (first in first out) manner so as to form the data segment.

3. The eliminator of claim 1, wherein when the data segment contains more than a half of waveforms of the eye blink artifact, the processing unit determines that the data segment contains the eye blink artifact.

4. The eliminator of claim 1, wherein when the sample entropy value is less than a predetermined threshold value, the processing unit determines that the data segment contains the eye blink artifact.

5. The eliminator of claim 1, wherein the processing unit outputs a value of zero as the output result for the data segment that contains the eye blink artifact, and the processing unit outputs the 96^{th} piece of the independent component data as the output result for the data segment that does not contain the eye blink artifact.

6. The eliminator of claim 1, wherein the detection unit starts to detect the eye blink artifact when a number of the pieces of the independent component data in the data segment reaches 128.

7. The eliminator of claim 1, wherein the independent component data received by the receiving unit are generated by an independent component analysis processor through an independent component analysis.

8. The eliminator of claim 1, wherein the processing unit sequentially performs the eye blink artifact elimination to data segments of multiple channels in the same time segment.

9. The eliminator of claim 8, wherein by performing an inverse independent component analysis, the processed independent component data outputted by the processing unit are converted into EEG signal data without the eye blink artifact.

10. The eliminator of claim 9, wherein the EEG signal data without the eye blink artifact are used for a subsequent EEG signal reconstruction.
